(19) United States
(12) Patent Application Publication
(10) Pub. No.: US 2016/0213320 A1
(43) Pub. Date: Jul. 28, 2016

(54) ORTHOPEDIC SUPPORT DEVICE AND ASSOCIATED METHODS

(71) Applicant: Abdo SHABAH, Montréal (CA)

(72) Inventor: Abdo Shabah, Montréal (CA)

(21) Appl. No.: 14/917,789

(22) PCT Filed: Sep. 9, 2014
(86) PCT No.: PCT/CA2014/050855

§ 371 Date: Mar. 9, 2016

Related U.S. Application Data
(60) Provisional application No. 61/875,382, filed on Sep. 9, 2013.

(51) Int. Cl. A61B 5/00; A61F 5/01; B29C 67/00; A61F 5/058

(52) U.S. Cl. CPC A61B 5/6812; A61B 5/4836; A61F 5/058; A61F 5/05841; A61F 5/0118; A61F 5/0111; B29C 67/0051; B29L 2031/753

(57) ABSTRACT

An orthopedic support device comprises a shell supporting a body portion; one or more stimulator provided on the shell for signaling a wearer of the orthopedic support device to perform a muscular activity, the one or more stimulator being adapted to communicate with a controller for controlling said one or more stimulator; and one or more sensor provided on an interior portion of the shell to capture biological activity data in relation to said body portion, the one or more sensor being adapted to store the biological activity data in a memory, the memory being in communication with the controller, in order to control the one or more stimulator on the basis of the biological activity data captured by the one or more sensor. Also disclosed is a method of engaging a body portion being supported by the orthopedic support device, and methods of making an orthopedic support device.

### FIELD OF THE INVENTION

The present invention relates to the field of orthopedic support systems, such as casts, braces and splints. More particularly, the present invention relates to orthopedic support devices, a method of engaging a body portion being supported by the orthopedic support device, and methods of making an orthopedic support device.

### BACKGROUND OF THE INVENTION

Known in the art are orthopedic casts for supporting a broken bone in a person's limb or part of a person's body until the bone has healed. Orthopedic casts are traditionally made by combining a cotton bandage with powdered calcined gypsum (also known as “plaster of Paris”) which is wetted to take the desired shape about the limb or other portion of the body, and hardens in place with the cotton bandage as it dries.

Also known are bandages of synthetic materials, such as knitted fiberglass bandages impregnated with polyurethane, or bandages of thermoplastic.

Such bandages must generally be kept on the limb or other body part for the entire duration of the healing process, which may take several weeks and several months in some cases. Indeed, the applying and removal of a conventional orthopedic cast requires specialized equipment and skill, usually from a medical facility. Moreover, a removed cast may not be remounted on the limb or other body portion. Once removed, a new cast must be made about the affected body portion, if necessary.

However, it is desirable for a patient wearing a cast to temporarily remove the cast, either for bathing, or for cleaning, airing and/or caring for the skin of the casted area, for example. There might also be medical indications to open temporarily the cast, like while travelling in high altitude and lowered pressure environments. It may also be desirable in some cases to interchange a cast for practical or esthetic purposes. For example, depending on particular situations, it may be desirable to wear a cast of a particular color, or having a pattern or the like. Moreover, depending on an activity being practiced, it may be desirable to wear a different format of cast. As an example, a lighter cast may be worn at rest, while a more robust and larger cast may be worn when walking, etc. In the same manner, a specific cast can be worn at rest, but a different one might be used during training or medical treatment. Moreover, it may desirable to interchange between a cast destined for indoor use and a cast destined for outdoor use. It may further be convenient to interchange casts depending on temperatures, exposure to water (for example when in the vicinity of a pool, or under rain), etc.

As stated at http//en.wikipedia.org/wiki/Orthopedic cast, the fact that the limb is unreachable during treatment causes the skin under the plaster to become dry and scaly because the discarded outer skin cells are not washed off. Also, plaster of Paris casts may lead to cutaneous complications such as macerations, ulcerations, infections, rashes, itching, burns, and allergic contact dermatitis. In hot weather, staphylococcal infection of the hair follicles and sweat glands may lead to severe dermatitis. Other limitations of plaster casts include their considerable weight and thus restricted movement. Furthermore, removal of the cast requires destroying the cast itself. The removal process requires the use of a special oscillating saw, which tend to be noisy. The special saw must be adapted to cut hard cast material but not soft material such as cast padding or skin. The removal may be distressing for the patient, especially children. Additionally, plaster of Paris casts tend to break down when exposed to water or the like.

Another challenge for patients wearing an orthopedic cast is lessened use of the muscle and diminished blood circulation, given the immobilization of the body member. Indeed, muscle exercises are recommended in order to avoid muscle atrophy. However, it is difficult for a healthcare professional to monitor the patient's exercises and sometimes for a patient to correctly execute the exercises.

In addition, different recommendations for a specific disease or cast use are sometimes difficult for patients to remember and/or follow, and a personalized follow-up is often required by healthcare professionals, which might be inconvenient for patients.

Present casting systems are traditional casts from either “plaster of Paris” or fiberglass. These systems are applied manually to a patient according to some guiding principles, as described in AAFP article—http://www.aafp.org/afp/2009/0101/p16.html.

Hence, in light of the aforementioned, there is a need for an improved system which, by virtue of its design and components, would be able to overcome some of the above-discussed prior art concerns.

### SUMMARY OF THE INVENTION

The object of the present invention is to provide a device which, by virtue of its design and components, satisfies some of the above-mentioned needs and is thus an improvement over other related engine control systems and/or methods known in the prior art.

An object of the present invention is achieved by an orthopedic support assembly and/or by an orthopedic support system, as will be easily understood, such as the one briefly described herein and such as the one exemplified in the accompanying drawings.

In accordance with an aspect, there is provided an orthopedic support device comprising: a shell for supporting a body portion to be healed; one or more stimulator provided on the shell for signaling a wearer of the orthopedic support device to perform a muscular activity, the one or more stimulator being adapted to communicate with a controller for controlling said one or more stimulator; and one or more sensor provided on an interior portion of the shell to capture biological activity data in relation to said body portion, the one or more sensor being adapted to store the biological activity data in a memory, the memory being in communication with the controller, in order to control the one or more stimulator on the basis of the biological activity data captured by the one or more sensor.

The “sensor” may be a pressure sensor, a temperature sensor (thermometer) and/or the like.

The “biological activity data” may be representative of a muscle contraction, a pulse, a temperature, a duration of the contraction, an intensity of the contraction, a frequency of contraction, a location of the contraction and a time of the contraction, and/or the like.

In a particular embodiment, the shell is made via three-dimensional printing. Moreover, at least a portion of each stimulator and at least a portion of each sensor is made integrally with the shell via said three-dimensional printing.

In particular embodiments, the controller and/or the memory may be mounted on the shell. Alternatively or additionally, the shell may be adapted to receive the controller and/or the memory. In some embodiments, the controller and/or memory may be provided by a smartphone device.

According to particular embodiments, the memory is configured to store a schedule and the controller is adapted to trigger the one or more stimulator based on the schedule. Alternatively or additionally, the orthopedic support device further comprises a geo-location module connected to the controller, in order to trigger the one or more stimulator based on a location of the orthopedic support device.

In a particular embodiment, the orthopedic support assembly further comprises a textile to be mounted between the shell and the body member. In a particular embodiment, the textile is mounted to an interior surface of the shell. In a particular embodiment, the one or more sensor is mounted onto the textile, or inside the shell, in order to provide a direct contact with the body portion.

According to particular embodiments, a circuit of sensors may be printed within the shell (via three-dimensional printing), in order to monitor the biological activity via the memory and controller. Further, the shell may include the input circuit (namely from the sensors) and an output circuit (for example, from transcutaneous electrical simulators). For example, at least a portion of each sensor and/or stimulator may be made integrally with the shell via three-dimensional printing.

In addition, the stimulator(s) may generate electrical pulses to contract the muscles. According to particular embodiments, the shell has a substantially tubular configuration for supporting a body portion to be healed, the shell comprising a resilient material, the shell being further operable between a default configuration wherein the shell is unbiased, and a biased configuration wherein the shell is expanded radially and compressed longitudinally.

According to particular embodiments, the shell substantially conforms to a shape of the body portion, the shell further comprising a relief along the interior portion to provide one or more pressure region in relation to an injury location in the body portion. At least one of said one or more sensor may be provided on the shell at each protrusion.

In a particular embodiment, the stored biological activity data may be used for analysis, reporting, sharing, comparing, etc. It is to be understood that the memory may be mounted on the support device and communicate with the one or more sensor locally (via a wire connection or wireless communication, such a Bluetooth™ for example, or other similar means). Alternatively, the memory may be located remotely and communicate with the one or more sensor via any suitable communication network. The memory may be part of or cooperate with a controller for processing the biological activity data before and/or after storage in the memory and for controlling said analysis, reporting, sharing, comparing, etc. The controller may be located locally or remotely with respect to either of the one or more sensor and the memory, as may be easily understood by a person skilled in the art.

Thus the orthopedic support device, the memory and/or the controller may be part of an orthopedic support system. It is to be understood that the communication between the one or more sensor, the memory and/or the controller may be substantially continuous, intermittent or on-demand. Is to be understood also that the memory and/or the controller may communicate with a plurality of sensors from different orthopedic casts, or any other digital device(s).

Advantageously, a patient wearing the orthopedic support assembly may perform exercises and the orthopedic support assembly captures biological activity data which may represent the contracting of muscles or the like, as well as duration, intensity, frequency, location and timing of the contractions.

Advantageously, the one or more stimulator may be configured to automatically and/or synchronically (notification or stimulates exercises such as transcutaneous electrical nerve stimulation (TENS) exercises or any equivalent) schedule exercises. The stimulators preferably cooperate with the sensors, for gathering the biological activity data in response to the stimulator action.

In a particular embodiment, the shell comprises multiple modules, the orthopedic support assembly further comprising:
one or more fastener for securing the modules of the orthopedic support device about the body portion.

In a particular embodiment, any of the one or more fastener is configured to operate between a fastening configuration wherein corresponding modules are positioned to support the body portion and a unfastening configuration wherein the corresponding modules are free from supporting the body portion.

Advantageously, the orthopedic support assembly may be temporarily removed and remounted repeatedly, as required. Preferably, the modules and fastener(s) are configured to allow the patient or a person assisting the patient to assemble or disassemble the orthopedic support assembly about the body portion, without requiring specialized skills i.e. without having to resort to a medical professional and/or having to travel to a clinic or the like.

Still advantageously, the orthopedic support assembly and/or components thereof may be interchangeable (for esthetic purposes or repair, etc.).

In a particular embodiment, the shell has graphical content printed on an exterior surface of the shell. It is to be understood that the shell may further comprises content printed on an interior surface of the shell (including specification data, patient related information, etc. or even a decorative design). The graphical content may conform with the surface of the shell. Alternatively, the graphical content or a portion thereof may comprise a relief. The graphical content may further be provided with fiber optic lighting. The graphical content may be a decorative design and/or alphanumeric characters or symbol providing information. Preferably a personalized graphical design is printed on the exterior surface of the shell.

Advantageously, the orthopedic support assembly may provide decorative features. Still advantageously, the orthopedic support assembly and/or components thereof may be interchangeable, for example to better fit esthetically with a patient's outfit for a particular occasion, or to change components for practical purposes based on a particular activity (for example to wear a waterproof cast when near water).

In accordance with another aspect, there is provided a method of engaging a body portion being supported by an orthopedic support device. The method comprises the steps of: signaling a wearer of the orthopedic support device to perform a muscular activity via one or more stimulator; capturing biological activity data in relation to said body portion via one or more sensor; storing the biological activity data in a memory; and triggering said one or more stimulator, via a controller based on the stored biological activity data.

According to embodiments of the method, the signaling step comprises at least one of: generating a mechanical movement (for example a vibration), generating an electric signal, outputting a sound, emitting a light, generating an output signal for a display screen, and/or the like. The signalling may be generated at at least one predetermined location of the body portion to target a particular muscle or muscle group. According to a particular embodiment, the memory comprises a schedule and the signalling is generated according to a schedule. The method may further include receiving geo-location information, and said the signalling may then be generated according to a particular geographical location of the orthopedic support device.

According to yet another aspect, there is provided an orthopedic support device comprising: a shell for supporting a body portion to be healed; and one or more sensor provided on the shell to capture biological activity data in relation to said body portion; wherein at least a portion of each sensor is made integrally with the shell via three-dimensional printing.

According to a particular embodiment, the above orthopedic support device further comprises one or more stimulator provided on the shell for stimulating the body portion, wherein at least a portion of each stimulator is made integrally with the shell via the three-dimensional printing.

The shell may comprise graphical content printed on an exterior surface of the shell. It is to be understood that the shell may further comprises content printed on an interior surface of the shell (including specification data, patient related information, etc. or even a decorative design). The graphical content may conform with the surface of the shell. Alternatively, the graphical content or a portion thereof may comprise a relief. The graphical content may further be provided with fiber optic lighting. The graphical content may be a decorative design and/or alphanumeric characters or symbol providing information. Preferably a personalized graphical design is printed on the exterior surface of the shell.

It is to be understood that according to embodiments, the shell may be made of multiple modular components.

In accordance with another aspect, there is provided a method of making an orthopedic support device comprising the steps of: providing a three-dimensional model of a body portion to be supported; generating a model of a shell of the orthopedic support device which conforms substantially with the shape of the body portion, said generating comprising defining in the model, at least one sensor location; and producing a shell of the orthopedic support device through three-dimensional printing of said model, including at least a portion of a sensor at each of said sensor location, through said three-dimensional printing.

The model of the shell is stored on a data storage.

According to an embodiment, the generating step further comprises defining in the model, at least one stimulator location, and wherein the producing comprises including in the shell, through said three-dimensional printing, at least a portion of a stimulator at each of said stimulator location.

It is to be understood that according to embodiments, the shell may be produced into multiple complimentary components.

According to yet another aspect, there is provided an orthopedic support device comprising: a shell having a substantially tubular configuration for supporting a body portion to be healed, the shell comprising a resilient material, the shell being further operable between a default configuration wherein the shell is unbiased, and a biased configuration wherein the shell is expanded radially and compressed longitudinally. This advantageously promotes bone alignment during a contraction of a muscle of the body portion.

According to an embodiment, the shell is made through three-dimensional printing. It is to be understood that according to embodiments, the shell may be made of multiple modular components.

According to particular embodiments, one or more sensor are mounted on an interior portion of the shell to contact the body portion, in order to capture biological activity data in relation to said body portion. At least a portion of each sensor may be made together with the shell through said three-dimensional printing. According to particular embodiments, the orthopedic support device further comprises one or more stimulator. At least a portion of each stimulator may be made together with the shell through said three-dimensional printing.

According to an embodiment, the shell has tapered extremities, each tapered extremity comprising an elastic material to allow inserting the body portion through the shell which has a tubular configuration.

The shell may comprise graphical content printed on an exterior surface of the shell. It is to be understood that the shell may further comprises content printed on an interior surface of the shell (including specification data, patient related information, etc. or even a decorative design). The graphical content may conform with the surface of the shell. Alternatively, the graphical content or a portion thereof may comprise a relief. The graphical content may further be provided with fiber optic lighting. The graphical content may be a decorative design and/or alphanumeric characters or symbol providing information. Preferably a personalized graphical design is printed on the exterior surface of the shell.

According to yet another aspect, there is provided an orthopedic support device comprising: a shell for supporting a body portion to be healed, the shell being made via three-dimensional printing, the shell substantially conforming to a shape of a body portion to be healed, the shell further comprising a relief along an inner surface to provide one or more pressure region in relation to an injury location in the body portion.

According to a particular embodiment, the relief comprises a protrusion, aligned substantially with the injury location, in order to exert pressure at the injury location. According to a particular embodiment, the relief comprises protrusion proximally and distally from the injury location to improve alignment of the body portion. According to embodiments, one or more sensor is mounted on an interior portion of the shell at each protrusion.

According to another aspect, there is provided a method of making an orthopedic support device comprising the steps of: providing a three-dimensional model of a body portion to be supported; providing location information of an injury in the body portion; generating a model of a shell of the orthopedic support device which conforms substantially with the shape of the body portion and further comprises relief based on the location information of the injury; and fabricating a shell of the orthopedic support device, through three-dimensional printing of said model.

According to embodiments, the generating may comprise at least one sensor position based on the relief. The fabricating may comprise including, through the three-dimensional printing, at least a portion of each of said at least one sensor.

According to yet another aspect, there is provided an orthopedic support device comprising: a shell for supporting a body portion to be healed, the shell being made through three-dimensional printing, the shell comprising personalized graphical content printed thereon.

According to a particular embodiment, the printed graphical content comprises a relief and/or fiber optic lighting.

According to a particular embodiment, the above orthopedic support device further comprises: one or more stimulator provided on the shell for signaling a wearer of the orthopedic support device to perform a muscular activity, the one or more stimulator being adapted to communicate with a controller for controlling said one or more stimulator; and one or more sensor provided on an interior portion of the shell to capture biological activity data in relation to said body portion, the one or more sensor being adapted to store the biological activity data in a memory, the memory being in communication with the controller, in order to control the one or more stimulator on the basis of the biological activity data captured by the one or more sensor.

Advantageously, the orthopedic support device is enhanced with an esthetic appeal and/or provides information regarding the patient, the support, etc.

It is to be understood that the above-described orthopedic support device with print may be combined with any compatible feature of the afore-mentioned orthopedic support assembly, orthopedic support system or modular orthopedic support device.

Other objects, advantages and features of the present invention will become more apparent upon reading of the following non-restrictive description of preferred embodiments thereof, given for the purpose of exemplification only, with reference to the accompanying drawings.

### BRIEF DESCRIPTION OF THE DRAWINGS

FIG. 1 is a perspective view of an orthopedic cast assembly, according to an embodiment of the present invention, shown mounted on a person's arm.

FIG. 2 is an exploded view of the orthopedic cast assembly shown in FIG. 1.

FIG. 3 is a flow chart representing steps of a method of manufacturing an orthopedic cast assembly, according to an embodiment of the present invention.

FIG. 4 is a perspective view of an orthopedic cast assembly, according to an embodiment of the present invention, the orthopedic cast assembly having a shell made of resilient material.

FIG. 5 is a perspective view of an orthopedic cast assembly, according to another embodiment of the present invention, the orthopedic cast assembly comprising a mounting means attached to the shell of the orthopedic cast.

### DETAILED DESCRIPTION OF PREFERRED EMBODIMENTS OF THE INVENTION

In the following description, the same numerical references refer to similar elements. The embodiments, geometrical configurations, materials mentioned and/or dimensions shown in the figures or described in the present description are preferred embodiments only, given for exemplification purposes only.

Broad Description of the Orthopedic Cast Assembly and System

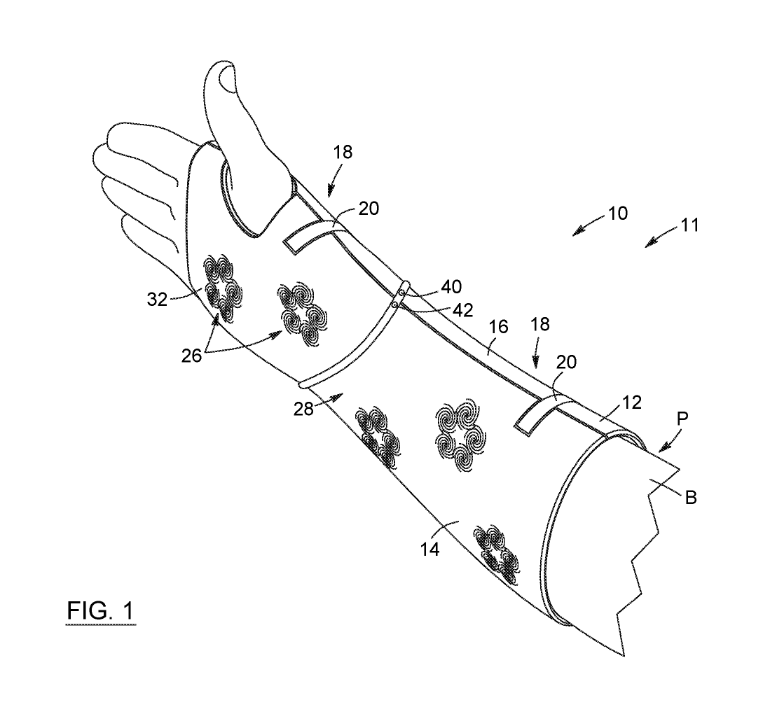

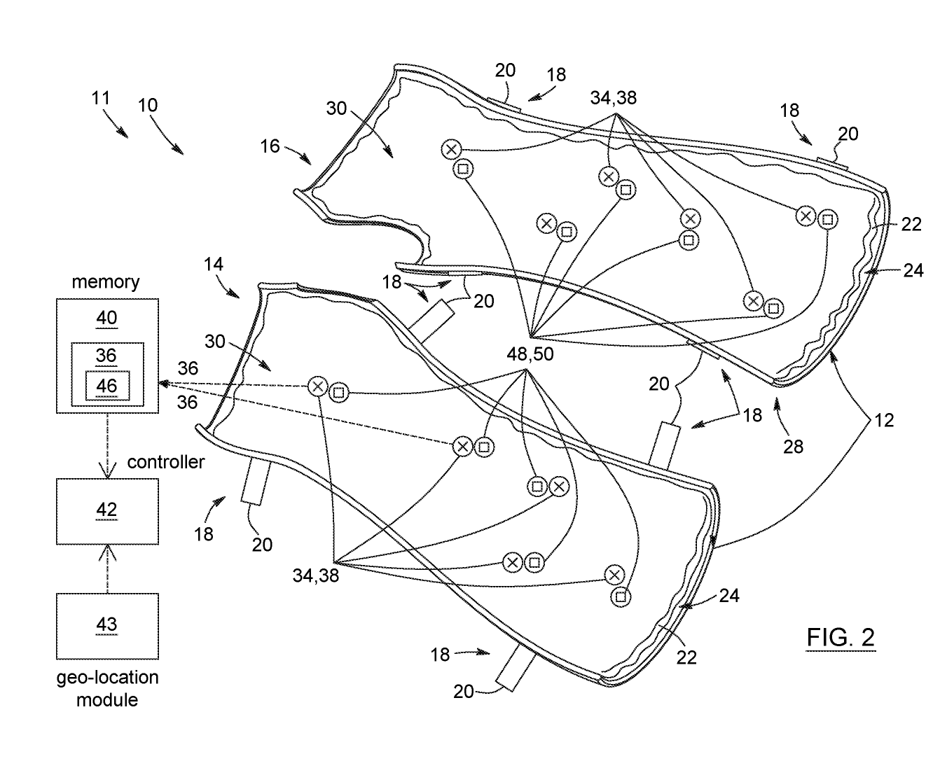

In accordance with an embodiment of the present, as shown in FIGS. 1 and 2, there is provided an orthopedic support assembly 11, namely an orthopedic cast assembly 10 which comprises a shell 12 to be placed about a body portion B to be healed, in order to support the body portion B.

The shell 12 is made up of modular components 14, 16 and a fastening system 18 which includes fasteners 20 for securing the modular components 14, 16 about the body portion B. The fasteners 20 are configured to operate between a fastening configuration wherein corresponding modules 14, 16 are positioned to support the body portion B and an unfastening configuration wherein the corresponding modules 14, 16 are free from supporting the body portion B.

In some embodiments, the unfastening configuration frees the modules from one another (i.e. unassembled configuration), while the fastening configuration binds the modules together (i.e. assembled configuration) about the body portion. In some embodiments, a fastener remains connected to the modules even in the unfastening configuration.

The fasteners 20 may be connectors as shown in FIGS. 1 and 2. Alternatively, the fastening system 18 may be provided by any other suitable component(s) such as an elastic band for example.

Advantageously, the orthopedic cast assembly may be temporarily removed and reinstalled repeatedly, as required. Preferably, the modules and fastener(s) are configured to allow the patient or a person assisting the patient to assemble or disassemble the orthopedic cast assembly about the body portion, without requiring specialized skills i.e. without having to resort to a medical professional and/or having to travel to a medical facility or the like.

Still advantageously, the orthopedic cast assembly 10 and/or modular components 14, 16 thereof may be interchangeable (for esthetic purposes, repair, change of size, change of shape, etc.).

It is to be understood that depending on the particular embodiment and the particular body member to be supported, the orthopedic cast assembly may be composed of any suitable number of modular components and fasteners, each of which may be provided in any suitable shape and size, as may be easily understood by the skilled reader.

The orthopedic cast assembly further comprises a textile 22 to be directly printed or positioned between the shell 12 and the body member B, for comfort and moisture absorption. In a particular embodiment, the textile 22 is mounted to an interior surface 24 of the shell 12.

The modular components 14, 16 forming the shell 12 further have graphical content 26 printed on an exterior surface 28 thereof.

The orthopedic cast 10 may thus be enhanced with an esthetic appeal and/or provides information regarding the patient, the cast, etc.

It is to be understood that an interior portion 30 (for example the textile 22) of the cast assembly 10 may also contain print, according to alternate embodiments.

It is to be understood that the shell may further comprises content (which may include specification data, patient related information, etc. or even a decorative design) printed on an interior surface 24 of the shell and/or on an interior portion 30 of the cast assembly 10.

The content printed on the interior 24, 30 or exterior 28 may be a decorative design and/or alphanumeric characters or symbol providing information. Preferably a personalized graphical design 32 is printed on the exterior surface 28 of the shell 12.

Thus, the orthopedic cast assembly 10 advantageously provides decorative features. Still advantageously, the orthopedic cast assembly 10 and/or components 14, 16 thereof may be interchangeable, for example to better fit esthetically with a patient's outfit for a particular occasion, or to change components for practical purposes based on a particular activity (for example to wear a waterproof cast when near water, etc.).

Sensors

Referring to FIG. 2, sensors 34 are mounted onto the textile 22, in order to enter into direct contact with the body portion B and capture biological activity data 36 in relation to the body portion B. The sensors 34 are positioned at different locations on the cast 10 in order to obtain activity data 36 of different muscles.

In an alternative embodiment, the sensors 34 or some of the sensors may be embedded in the shell 12 of the support device 11.

It is to be understood that any number of such sensors 34 may be provided depending on the particular embodiment (for example to obtain average information from multiple sensors, to obtain precise local information, to obtain different types of information, to provide back-up in case of failure of one sensor, etc.)

A “sensor” 34 may be a pressure sensor, a temperature sensor (thermometer) and/or the like. The “biological activity data” may be representative of a muscle contraction, a pulse, etc.

In this particular embodiment shown in FIGS. 1 and 2, the sensors 34 are a pressure sensors 38 and essentially capture muscle contractions.

The orthopedic cast assembly 10 further comprises a memory 40 which communicates with the sensors 34 (via a wire connection or wireless communication, such a Bluetooth™ for example, or other suitable means), in order to store the biological activity data 36 thereon. The stored data 36 may then be used for analysis, reporting, sharing, comparing, etc.

Moreover, the orthopedic cast assembly 10 comprises a controller 42 for processing the biological activity data 36 before and/or after storage in the memory 40 and for controlling said analysis, reporting, sharing, comparing, etc. The orthopedic cast assembly 10 further comprises a geo-location module 43.

It is to be understood that according to alternate embodiments, a memory may be located remotely and communicate with the sensors via any suitable communication network. Moreover, a controller may be located locally or remotely with respect to either of the sensors and the memory, as may be easily understood by a person skilled in the art.

Thus the orthopedic cast assembly 10, the memory 40 and/or the controller 42 may form part of an orthopedic cast system 44. It is to be understood that the communication between the sensors 34, the memory 40 and/or the controller 42 may be substantially continuous, intermittent or on-demand.

Is to be understood also that, according to particular embodiments, a memory and/or a controller may communicate with a plurality of sensors from different orthopedic casts.

Advantageously, the orthopedic cast assembly 10 captures, by means of the sensors 34, the biological activity data 36, namely muscle contraction information 46, in relation to exercises performed by a patient P wearing the orthopedic cast assembly 10. The muscle contraction data 46 may include duration, intensity, frequency, location and time and date of the contractions, and is stored in the memory 40.

The stored data 46 may further be processed for example via a computer program, to produce a corresponding report.

According to some embodiments, biological activity data may be gathered from multiple memories (relative to multiple casts or cast wearers), for reporting, etc.

Advantageously, a physician or other health professional may obtain a report of the exercises having been performed by the patient (or any other monitored physical activity, voluntary or involuntary). This information may be correlated with the patient's progress, as well as with other patient's exercises (or biological activity data) and/or progress. The patient may further broadcast or send the biological activity data over a communication network to share the information or direct the information to the health professional, etc.

Muscle Stimulation

Referring still to FIG. 2, the orthopedic cast assembly 10 further comprises signal generators 48 mounted on the orthopedic cast, for signaling the wearer P of the cast 10 to perform a biological activity, namely a muscle contraction in the present embodiment.

The “signal” generated by the signal generator may be an electric signal, a mechanical movement, a sound, a light, an indication on a display screen, and/or the like, depending on the particular embodiments.

According to the present embodiment, a mechanical movement is generated by electro-mechanic components 50 mounted on the interior portion 30 of the cast 10. The electro-mechanic components 50 are placed at different locations on the interior 30 of the cast 10 to be directed to different muscles to be exercised.

The electro-mechanic components 50 are controlled by the controller 42. The controller 42 may trigger the electro-mechanic component 50 to generate a signal based on a predetermined schedule stored in the memory 40. Thus, a signal may be repeated sequentially over a duration period (for example, every 2 seconds over a period of one minute). This sequence may in turn be scheduled to be repeated at various times during the day.

Advantageously, the patient is automatically reminded or even physically stimulated, by means of the cast 10, in accordance with some embodiments, to perform exercises, in order to resist muscle atrophy and promote blood circulation.

The signal generators preferably cooperate with the sensors 34, for gathering the biological activity data 36 in response to the stimulation action of the electro-mechanic components 50. And further send stimulation signals in response to the biological activity data 36 gathered (based on the specific locations of each sensor 34 and signal generator 48).

Manufacturing of the Orthopedic Cast

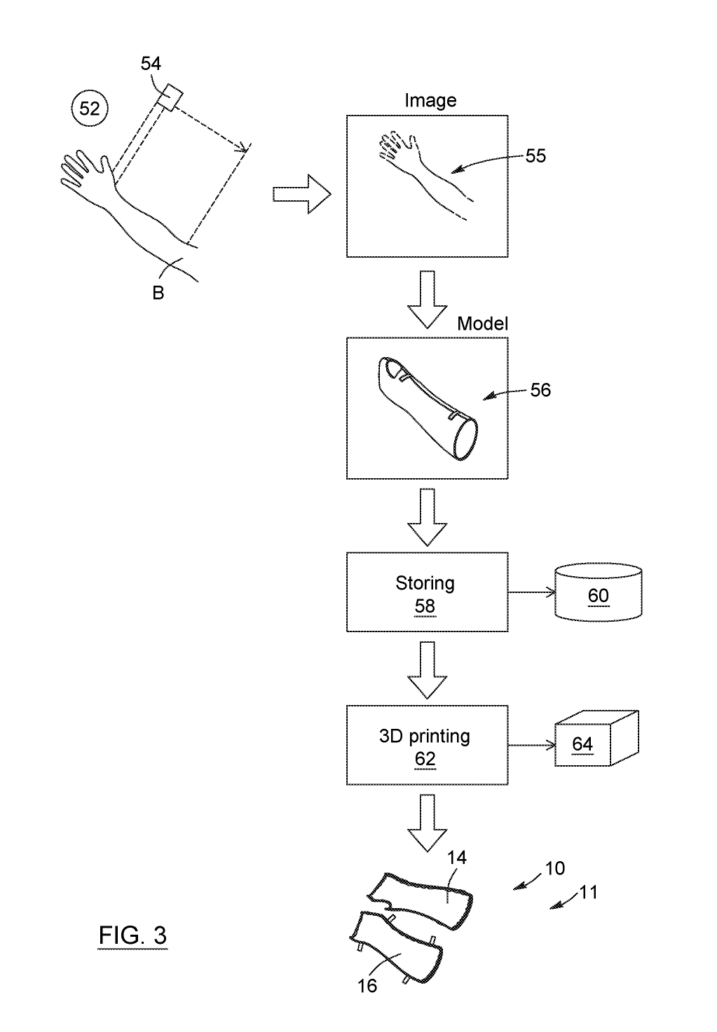

Referring now to FIG. 3, the above-described orthopedic cast assembly 10 is manufactured by:
capturing (at step 52), by means of a sensor system 54, a three-dimensional image 55 of the body portion B;
storing (at step 58) the image 55 in a storage unit 60;
generating in the storage unit 60 a model 56 of a shell of an orthopedic cast 10 to be mounted about the body portion B, based on the captured image 55;
constructing (at step 62), by means of a three-dimensional printer 64, the modular components 14, 16 to form the shell 12 of the orthopedic cast 10, based on the model 56 having been captured and stored;
providing attachment means 20 to the modular components 14, 16 to mount the orthopedic cast 10 about the body portion B (each fastener 20 may be mounted to one or more of the modules 14, 16 or may be provided separately therefrom when in the unfastening configuration) (see FIG. 2);
installing a textile 22 on the interior surface 24 of each modular component 14, 16 (see FIG. 2);
printing a print 32 on an exterior surface 28 of the orthopedic cast 10 (the print may comprise graphical print and/or alphanumeric print, which may provide esthetic appeal and/or information) (see FIG. 2);
mounting the sensors 34 on the orthopedic cast 10, on the interior portion 30 of the orthopedic cast 10 (see FIG. 2); and
mounting the signal generators 48 on the interior portion 30 of the orthopedic cast 10 (see FIG. 2).

It is to be understood that the above-listed steps may be performed in any suitable order, as may be easily understood by a person skilled in the art, depending on particular embodiments of the present. Furthermore, some of the steps may be executed simultaneously, as may also be readily understood.

The three-dimensional printing, at step 62, is performed with a suitable plastic or fiberglass material, and/or any other material having a suitable weight, flexibility and durability.

The method allows a clinician to shape the 3D modeled cast based on their clinical preferences, via a touch screen for example.

The above-described method uses an image (X-ray (XR) or CT-SCAN, ultrasound or MRI or any kind of image or any combination allowing visualization of boney structures) and 3D printing, using the Sir John Charnley method to develop a system of cast. Using a specific algorithm that allows a level of material extrusion of cast (heightening outward), but once installed, with low pressure will create an intrusion at the brace to strengthen the specific splint. The algorithm used to create the extrusion defines a strategic region to apply pressure just for appropriate and clinically better healing. These pressure points may be applied to the fracture site in some cases, as well as proximal than distal to the injury, to provide better alignment. This is useful for better reduction and healing of the injury, but also to allow a better stabilisation of the device once installed. The pressure point could serve to stabilize the cast, even if pressure points are applied on normal parts of the body. In that manner, the device could be easily weared as it is designed to be larger of the body portion it needs to hold, but because of this method could stabilize the device on specific pressure points.

The bone picture provides a visualization of the fracture. Benchmarks (radio-opaque lines in the limb affected, such as a radio-opaque simple step) allow better positioning articulation, fracture, and ensure proper calculation in the algorithm (which could use these benchmarks in its calculation, or simply the image) in conjunction with the 3D image scans. It will also allow, in conjunction with the 3D rendering (3D volume and color of the body part), to estimate through a specific algorithm the edema on the body part, and then render the best shape that the 3D model should be, taking into account the decreased edema-swelling expected in the next days. This helps the reuse of the orthopedic support system in the context of decreased swelling.

It is to be understood, that according to alternative embodiments, 3D scanning may be used in combination with a 2D or X-Ray scan, if the scan device permits it. It is assumed that if 3D bone reconstruction are available (via Computed Tomography (CT) Scan, for example), this could substitute the X-Ray for better cast modeling. In alternative embodiments, magnetic resonance imaging (MRI) or ultrasound, or any combination of the above may also be used.

Thus, the above-described method comprises, prior to the constructing (at step 62), steps of:
scanning the body portion B, for example using XR, CT SCAN, MRI, ultrasound or any other method, to locate one or more region in the body portion B to be treated (for example, locating a bone fracture using XR, CT Scan, MRI, ultrasound and/or any combination thereof); and
adjusting the shape of the model 56 based on the located region(s) (for example by raising or lowering the relief of the model 56, in accordance with the above-described Sir John Charnley method) to provide additional pressure and/or support in relation to the located region(s) of the body portion B to be treated.

Other Features

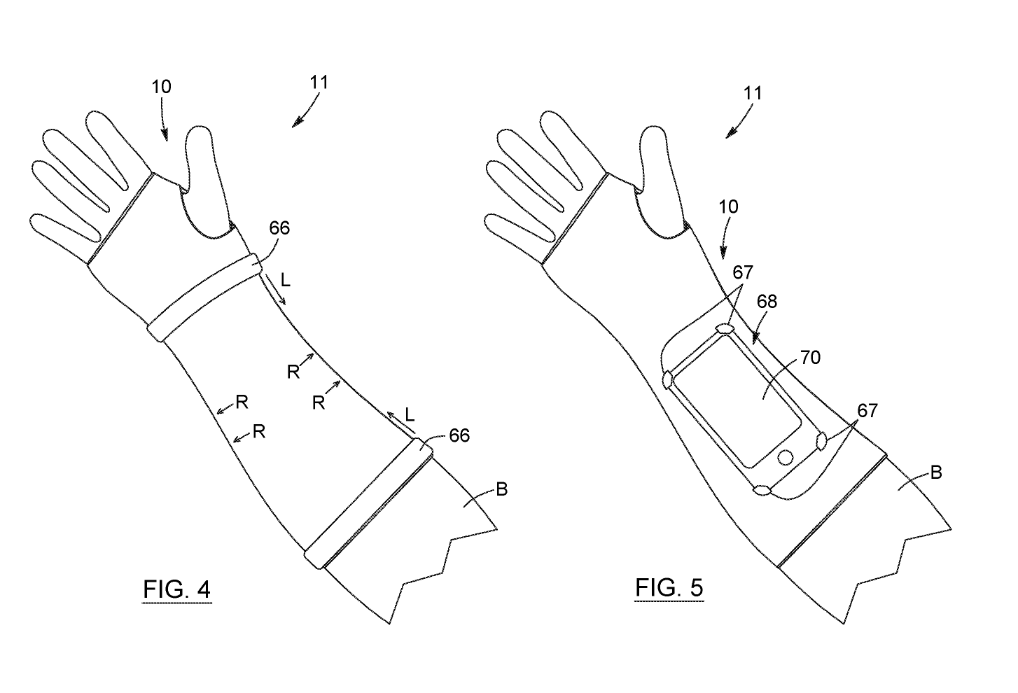

According to embodiments of the present invention, with reference to FIG. 4, the shell 12 of the orthopedic cast assembly 10 comprises a resilient material in order to contract in the longitudinal direction L when the shell 12 is expanded radially in the direction R, for example when the cast wearer makes muscle contractions, and to return to its original configuration when the radial pressure from the muscle contraction is released. The periodic contraction of the shell 12 in the longitudinal direction L promotes bone growth and thus the healing of the limb. The longitudinal contraction is progressive and small in range so as to prevent bone disalignment. Preferably, the shell comprises attachment components 66 providing pressure points which are displaced longitudinally toward each other when the radial expansion occurs. As shown in FIG. 4, the attachment components 66 are located toward opposite extremities of the shell 12. It is also assumed that the multi-part cast system could also include a removable part, and that constant longitudinal pressure could be provided by a resilient material to have permanent pressure for bone growth improvement.

According to embodiments of the present invention, with reference to FIG. 5, the orthopedic cast assembly 10 comprises a mounting means 67 for receiving a smart phone 68, other handheld computer device, telephone or an electronic display device. The mounting means may be made integrally with the shell 12 for example, or it may be provided as an add-on component or system.

According to an embodiment, the smart phone 68 provides a controller and/or a memory for operating the electro-mechanic components 50 and collect data from the sensors 34. Still in accordance with an embodiment, the smart phone 68 serves as a vibration actuator 70 for periodically vibrating the cast assembly 10. The vibration may be initiated in accordance with a predetermined schedule, and/or based on additional information (such as history, geographical location, time, user setting to momentarily avoid vibrations, etc.), which is stored and/or computed by the smart phone 68. It is to be understood that the orthopedic cast assembly 10 may alternatively have mounted thereon, an integrated vibration actuator which is controlled by the controller 42 (see FIG. 2). This vibration system is advantageous in that periodic vibrations promote bone growth and thus promote repair of the wounded limb.

According to an embodiment, the controller 42 and memory 40 (see FIG. 2) are adapted to provide geo-location capabilities, in order to take geographical location into consideration for triggering the electro-mechanic components 50. For example, the cast system may be adapted to only prompt the wearer to perform exercises (by triggering the electro-mechanic components 50) when the wearer arrives home, i.e. not at the workplace, or it might be activated during a flight after reaching an airport and during take off. Thus, the orthopedic cast assembly 10 comprises a gps device or other location detection system.

In addition, the orthopedic cast assembly, according to an embodiment, comprises a gyroscope, a pedometer or the like, in order to determine the displacement produced by the cast wearer and to correlate the displacement with exercises having been performed by the cast wearer independently of the cast system described herein. The cast system thus determines an amount and/or type of exercise to be prompted by the cast system depending on the displacements made.

The orthopedic cast assembly, according to the embodiment shown in FIGS. 1 and 2, is a multi-modular system. The shell 12 comprises a flexible material and a provision of more ‘malleable’ modules allows such a cast to be adapted as orthoses to be used in case of sprains, i.e. ligament damage, instead of bone fracture. The system thus allows better stabilization of the targeted joint. In the context of an orthosis, the sensors and stimulation modules may be solicited for improved pain relief after training. This system for sprains may also be cooled so as to further promote pain relief.

In addition, the orthopedic cast assembly, according to an embodiment, comprises an altimeter, so as to expand the shell 12 (see FIG. 2), for example by separating the modular components 14, 16 from one another, depending on a detected altitude. This feature may be particularly advantageous when a wearer flies on an aircraft.

In accordance with an alternate embodiment, the cast is provided with openings in appropriate regions of the cast, in order to lessen the weight of the cast.

Advantageously, the orthopedic cast and/or components thereof may be manufactured quickly and remotely, and then shipped to the patient or to a clinic in proximity to the patient.

Still advantageously, the capability for health professionals to monitor and recommend particular exercises for improved healing (automated self-care), and socially connect cast wearers with specialized help (a healthcare provider) or socially with a community of other users targeted to a specific injury or disease, may also be helpful to improve the healing process (guided care through expert advice or social network recommendations).

Various aspects of the invention will now be described, in accordance with particular embodiments.

According to a particular embodiment, there is provided an orthopedic support assembly, such as a cast, orthesis, brace or splint assembly, or any other orthopedic support assembly, comprising a shell for supporting a body portion to be healed; and one or more sensor mounted on an interior portion of the shell to contact the body portion, in order to capture biological activity data in relation to said body portion.

According to another aspect of the present invention, there is provided a modular orthopedic support assembly comprising: modular components configured to form a shell about a body portion for supporting said body portion; and one or more fastener (i.e. a fastening system) for securing the modular components about the body portion.

Advantageously, the support may be easily removed and reinstalled about the body portion. It is to be understood that the above-described modular orthopedic support may be combined with any compatible feature of the afore-mentioned orthopedic support assembly, device or system.

According to another embodiment, there is provided a method of manufacturing an orthopedic support device for supporting a body portion to be healed, comprising the steps of:
providing a three-dimensional model of the body portion (for example, by capturing an image of the body portion by means of a sensor system, or obtaining a precaptured or predefined 3D model of the body portion
adjusting the model for the patient based on approximation, direct measurements or a sensor system), preferences or judgement; and
performing a three-dimensional printing of the model having been captured, to construct the orthopedic support device.

The “sensor system” may include an optical sensor, an acoustic sensor, a laser scanning sensor, and/or the like, as well as a memory for storing the data relating to the three-dimensional model.

In a particular embodiment, the three-dimensional printing is performed with plastic, fiberglass, or any particular material having suitable degrees of flexibility, durability, water and heat resistance, and ease of manufacturing and integration of monitoring and stimulation technology.

In a particular embodiment, the three-dimensional printing comprises printing modular components, and the method further comprises providing attachment means on the modular components to mount the orthopedic support device about the body portion.

In a particular embodiment, the method further comprises printing a print on an exterior surface of the orthopedic support device. The print may comprise graphical print and/or alphanumeric print, which may provide esthetic appeal and/or information.

In a particular embodiment, the method further comprises direct printing or mounting one or more sensor on the orthopedic support device, preferably on an interior portion of the orthopedic support device, for capturing biological activity of the body portion.

In a particular embodiment, the method further comprises direct printing or mounting one or more signal generator on the orthopedic support device, preferably on an interior portion of the orthopedic support device, for signaling a wearer of the support device to exercise the affected body portion, and/or to stimulate directly a targeted muscle.

Advantageously, the orthopedic support device and/or components thereof may be manufactured remotely and shipped to the patient or to a clinic in proximity to the patient.

According to another embodiment, there is provided a method of monitoring biological activity of a wearer of an orthopedic support device, the method comprising the step of: capturing biological activity data, by means of a sensor mounted on the orthopedic support device; and storing the biological activity data in a memory.

The step of capturing may be performed by a pressure sensor, a temperature sensor, and/or the like. The sensor may be mounted on an interior portion of the orthopedic support device so as to come in contact with the body of the wearer. The biological activity data may be representative of a muscle contraction, a pulse, etc. and may include the date/time, location, intensity level, etc of each muscle contraction, pulse, etc.

According to a particular embodiment, the method may further comprises processing the stored biological activity data to produce a corresponding report.

According to a particular embodiment, the method may further comprises gathering biological activity data from multiple memories (which may come, for example, from multiple casts or cast wearers, or from other wearable device(s)) to produce a corresponding report.

Advantageously, a physician or other health professional may obtain a report of the exercises having been performed by the patient (or any other monitored physical activity, voluntary or involuntary). This information may be correlated with the patients progress, as well as with other patient's exercises (or biological activity data) and/or progress. The patient may further broadcast or send the biological activity data over a communication network to share the information or direct the information to the health professional, or a community of users, etc.

According to another embodiment, there is provided a method of stimulating a supported body member, comprising the steps of: generating a signal, by means of a signal generator mounted on an orthopedic support device, for signaling the wearer of the support device to perform an exercise, and/or to stimulate directly a targeted muscle.

The “signal” be an electric signal, a mechanical movement, a sound, a light, an indication on a display screen, and/or the like.

According to a particular embodiment, a mechanical movement is generated by an electro-mechanic component mounted on an interior of the support device. Such an electro-mechanic component may be placed at different locations on the interior of the support device to be directed to particular muscles to be exercised.

According to a particular embodiment, the step of generating the signal is triggered by means of a controller. The step of generating the signal may be repeated, by means of the controller, based on a predetermined schedule stored in a memory. The step of generating the signal may be repeated sequentially over a duration period (for example, every 2 seconds over a period of one minute). This sequence may in turn be scheduled to be repeated at various times during the day.

Advantageously, the patient is automatically reminded or even physically stimulated, by means of the support device, in accordance with some embodiments, to perform exercises, in order to resist muscle atrophy and promote blood circulation.

It is to be understood that the above-described method of stimulating a supported body member may be further combined with any compatible features of the afore-mentioned method of monitoring biological activity of a wearer of the support device.

It is to be understood that although the embodiments described are directed to orthopedic casts or orthopedic cast systems, the present invention may also be applicable to a splint, brace and/or any other suitable devices for controlling the movement of articulations and/or supporting fractured bones, either flexible or rigid. Moreover, although the preferred embodiment described herein and illustrated in the accompanying drawings comprises components such as a textile, a pressure sensor, a memory, a controller, etc., and although the associated method include steps as explained and illustrated herein, not all of these components, configurations and steps are essential to the invention and thus should not be taken in their restrictive sense, i.e. should not be taken as to limit the scope of the present invention. It is to be understood, as also apparent to a person skilled in the art, that other suitable components and cooperations thereinbetween, as well as other suitable configurations, organizations and/or architectures may be used for the vehicle acceleration control module according to the present invention, as will be briefly explained herein and as can be easily inferred herefrom, by a person skilled in the art, without departing from the scope of the invention. Moreover, the order of the steps provided herein should not be taken as to limit the scope of the invention, as the sequence of the steps may vary in a number of ways, without affecting the scope or working of the invention, as can also be understood.

The above-described embodiments are considered in all respect only as illustrative and not restrictive, and the present application is intended to cover any adaptations or variations thereof, as apparent to a person skilled in the art. Of course, numerous other modifications could be made to the above-described embodiments without departing from the scope of the invention, as apparent to a person skilled in the art.

1. An orthopedic support device comprising:
a shell for supporting a body portion to be healed;
one or more stimulator provided on the shell for signaling a wearer of the orthopedic support device to perform a muscular activity, the one or more stimulator being adapted to communicate with a controller for controlling said one or more stimulator; and
one or more sensor provided on an interior portion of the shell to capture biological activity data in relation to said body portion, the one or more sensor being adapted to store the biological activity data in a memory, the memory being in communication with the controller, in order to control the one or more stimulator on the basis of the biological activity data captured by the one or more sensor.

2. The orthopedic support device according to claim 1, wherein the shell is made via three-dimensional printing.

3. The orthopedic support device according to claim 1, wherein
at least a portion of each stimulator and at least a portion of each sensor is made integrally with the shell via three-dimensional printing.

4. The orthopedic support device according to claim 1, further comprising said controller and memory mounted on the shell.

5. The orthopedic support device according to claim 1, wherein the shell is further adapted to receive said controller and memory.

6. The orthopedic support device according to claim 5, wherein the controller and memory are provided by a smartphone device.

7. The orthopedic support device according to claim 4, wherein the memory is configured to store a schedule, the controller being adapted to trigger the one or more stimulator based on the schedule.

8. The orthopedic support device according to claim 1, further comprising a geo-location module connected to the controller, in order to trigger the one or more stimulator based on a location of the wearer of the orthopedic support device.

9-16. (canceled)

17. A method of engaging a body portion being supported by an orthopedic support device, the method comprising the steps of:
signaling a wearer of the orthopedic support device to perform a muscular activity via one or more stimulator;
capturing biological activity data in relation to said body portion via one or more sensor;
storing the biological activity data in a memory; and
triggering said one or more stimulator, via a controller based on the stored biological activity data.

18-20. (canceled)

21. The method according to claim 17, further comprising receiving geo-location information and wherein the signalling is generated according to a particular geographical location of the orthopedic support device.

22-28. (canceled)

29. An orthopedic support device comprising:
a shell having a substantially tubular configuration for supporting a body portion to be healed, the shell comprising a resilient material, the shell being further operable between a default configuration wherein the shell is unbiased, and a biased configuration wherein the shell is expanded radially and compressed longitudinally.

30. The orthopedic support device according to claim 29, wherein the shell is made through three-dimensional printing.

31. The orthopedic support device according to claim 29, further comprising one or more sensor mounted on an interior portion of the shell to contact the body portion, in order to capture biological activity data in relation to said body portion.

32. The orthopedic support device according to claim 31, wherein at least a portion of each sensor is made together with the shell through said three-dimensional printing.

33. The orthopedic support device according to claim 29, further comprising one or more stimulator.

34. The orthopedic support device according to claim 33, wherein at least a portion of each stimulator is made together with the shell through said three-dimensional printing.

35. The orthopedic support device according to claim 29, wherein the shell has tapered extremities, each tapered extremity comprising an elastic material to allow inserting the body portion through the shell.

36. The orthopedic support device according to claim 29, further comprising graphical content printed on the shell.

37. The orthopedic support device according to claim 36, wherein the printed graphical content comprises at least one of: a relief and fiber optic lighting.

38. An orthopedic support device, comprising:
a shell for supporting a body portion to be healed, the shell being made via three-dimensional printing, the shell substantially conforming to a shape of a body portion to be healed, the shell further comprising a relief along an inner surface to provide one or more pressure region in relation to an injury location in the body portion.

39. The orthopedic support device according to claim 38, wherein the relief comprises a protrusion, aligned substantially with the injury location.

40. The orthopedic support device according to claim 38, wherein the relief comprises protrusion proximally and distally from the injury location to improve alignment of the body portion.

41. The orthopedic support device according to claim 38, further comprising one or more sensor mounted on an interior portion of the shell at each protrusion.

42-47. (canceled)
